(19) United States
(12) Patent Application Publication
(10) Pub. No.: US 2010/0234683 A1
(43) Pub. Date: Sep. 16, 2010

(54) THERAPEUTIC-SEXUAL APPLIANCE FOR MEN

(76) Inventors: Jozsef Vizkeleti, Szolnok (HU); Lajos Kalman, Torokszentmiklos (HU)

Correspondence Address: DOWELL & DOWELL P.C., 103 Oronoco St., Suite 220, Alexandria, VA, 22314, US

(21) Appl. No.: 12/448,218

(22) PCT Filed: Dec. 1, 2006
(86) PCT No.: PCT/HU2006/000106

§ 371 Date: Jun. 12, 2009

(30) Foreign Application Priority Data
Dec. 13, 2005 (HU) P0501151

(51) Int. Cl. A61F 5/00

(52) U.S. Cl. 600/39

(57) ABSTRACT

The invention relates to a therapeutic-sexual appliance for men, which is self-supporting, hollow, and has elastic wall. The characteristics of the appliance are that it has a penis holder (1) having shape and surface similar to the penis, ending with a rounded point, a scrotum holder (2) connected to it by a continuous material transition provided with an elliptic supporting opening (3) on the upper side and a scrotum passing opening (4) on the lower side.

The invention relates to a therapeutic-sexual appliance for men having problems with sexual functions.

The components of the man's sexual function are the adequate sexual lust (libido), the tumescence of the penis of adequate strength and duration under influence of sexual excitation (erection), as well as the ejaculation followed by orgasm.

The disorder of any component can lead to sexual dysfunction

The sexual life in sexual relations is the most important indicator of the physical and spiritual health and the quality of life.

One speaks about tumescence disorder, potency disorder, erection disorder or erection dysfunction with generally used physician's expression in case of long-lasting inability to reach and/or keep the erection necessary for sexual activity.

The widely known expression of impotency is the name for the most severe form of all kinds of the erection disorder.

The erection disorder is a disease occurring very frequently getting more and more severe by the advance of age and affects a great number of men all over the world.

The erection disorders may have either psychic or organic causes.

The most important risk factors of the erection disorders are the age, the chronic diseases (diabetes, high blood pressure, diseases of coronary artery), in addition to them the depression, a number of medicines taken against chronic diseases and their side-effects as well as stimulants such as smoking and alcohol.

In addition to the above mentioned causes there are some diseases of the nervous system, surgical interventions in the lesser pelvis, which can damage the nerves, which cause sexual damages.

The sexual act becomes impossible because of the insufficient erection, as the erection cannot be simulated.

The treatment with medicaments is a relatively new process for the treatment of erection disorders, where a medicament taken in short time before the act can help to come the erection into existence, but it acts only temporarily and does not result in a final improvement because of its numerous side effects.

The protection against infections and the protection against undesired pregnancy are problems of the sexual relations at the same time.

The condom used everywhere protects, however, against the infections and undesired pregnancy but it cannot be used if the erection of the penis is not adequate.

The published international patent application of No WO 8806432 describes a tube-shaped contraceptive for prevention of infections propagated by sexual intercourse.

This appliance consists of a tube provided with a collar on its open end, which could be held in stressed state. The diameter of the tube is large enough to allow the movement of the penis therein and the closed end of the tube is provided with an element, which provides its fastening on the uterus.

The disadvantage of the appliance is that its application is complicated it does not provide the appropriate feeling of comfort and its fastening is uncomfortable.

There are appliances to replace penis, which may be bound onto the man's body in a complicated way then used.

The disadvantages of the known appliances are that they may be used either at a certain state of erection or their binding is complicated and destroys illusions.

We set the aim to develop a therapeutic-sexual appliance, which is free of the above mentioned disadvantages and which may be applied completely successfully even in the case of complete impotence.

The therapeutic-sexual appliance, which we developed, makes the penis sufficiently stiff for the intercourse so that the partners have complete excitation of the terminal nerves during the sexual intercourse.

The therapeutic-sexual appliance for sexual disorder developed by us is self-supported, it has a cavity inside and elastic wall.

The cavity in the appliance enables to put the penis inside and has such a shape that no separate fastening apparatus is necessary but makes the feeling of the body and also enables to increase the lust.

The erection problem may be resolved by its usage so that the person using it does not need to give up the usage of his own organ.

The invention relates to a self-supported therapeutic-sexual appliance for men having sexual disorder with a cavity inside and elastic wall.

The characteristics of the appliance are that it has shape and surface similar to the penis, having a penis holder ending with a rounded point on its one side and a scrotum holder connected to it by continuous material transition and having an elliptic supporting opening on the upper side and an opening to let the scrotum passing on the lower side.

A possible embodiment of the appliance is where the outer surface of the penis holder is provided with regularly or irregularly distributed protuberances.

Another possible embodiment of the appliance is where the penis holder is provided with a penis ring at the root of the penis.

Any of the above mentioned embodiments may be shaped in such a way that the inner surface of the penis holder is provided with regularly or irregularly distributed ribs.

The invention relates also to a such version of any above mentioned embodiment of the appliance where the internal surface of the penis holder is provided with a longitudinal urethra holder groove.

The invention relates also to such an embodiment where the inside of the penis holder is provided with a vibrator holder in the part ending with rounded point and the inside of the scrotum holder is provided with a battery holder.

The invention relates to such versions of all the above mentioned embodiments where the penis holder is provided with a hole on the end having rounded point to let the seminal fluid out.

Another advantageous variant of any of the above mentioned embodiments is where the penis holder is provided with a bulge on its outer surface at the root of the penis to excite the clitoris.

Any construction containing any combination of all the above mentioned characteristics may be developed, which are all still in the scope of the invention so the penis holder of the appliance may be provided either with regularly or irregularly distributed protuberances and/or with penis ring and/or internal ribs and/or longitudinal urethra holder groove and/or vibrator holder and/or outlet hole for seminal fluid and/or with clitoris exciting bulge.

The wall of the therapeutic-sexual appliance is advantageously made of silicon rubber.

An embodiment is also applicable where the elastic wall of the medical appliance for sexual disorder is made of a layered material wherein a plastic net is placed between two silicon rubber layers.

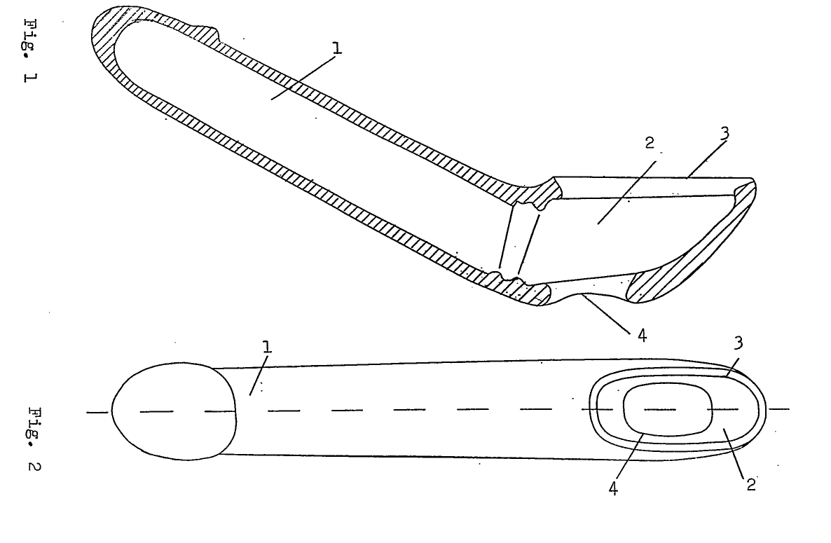

The construction according to the invention is shown in the FIGS. 1 to 11.

The FIG. 1 shows the front view of an embodiment of the appliance in longitudinal section and

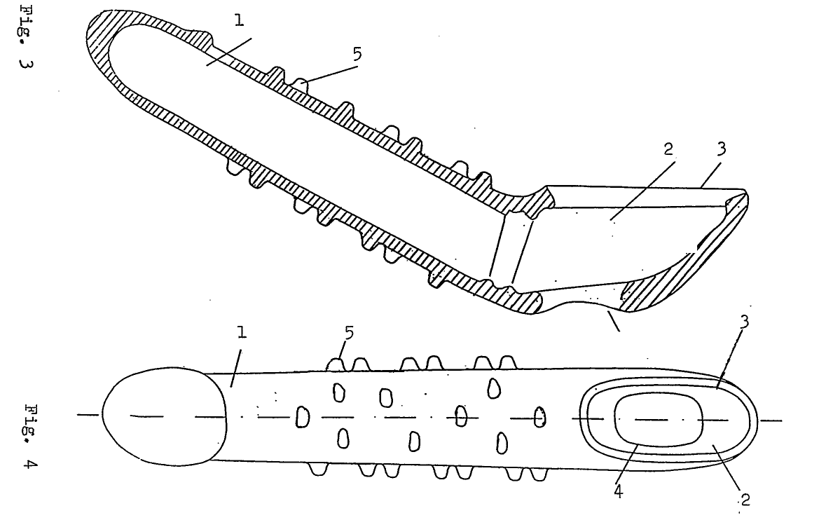

The FIG. 2 shows the top view of the appliance in the FIG. 1.

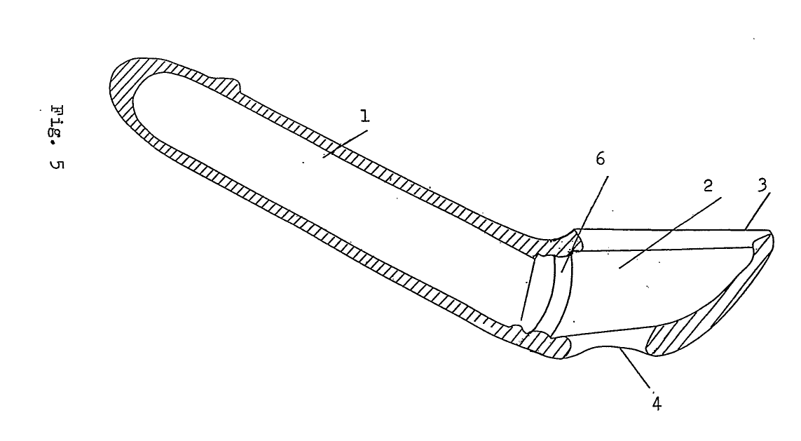

The FIG. 3 shows the front view of the embodiment of the appliance provided with special outer surface in longitudinal section and

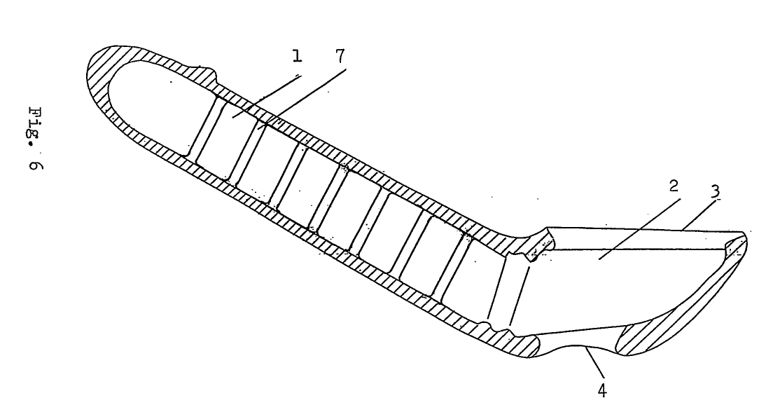

The FIG. 4 shows the top view of the appliance in the FIG. 3.

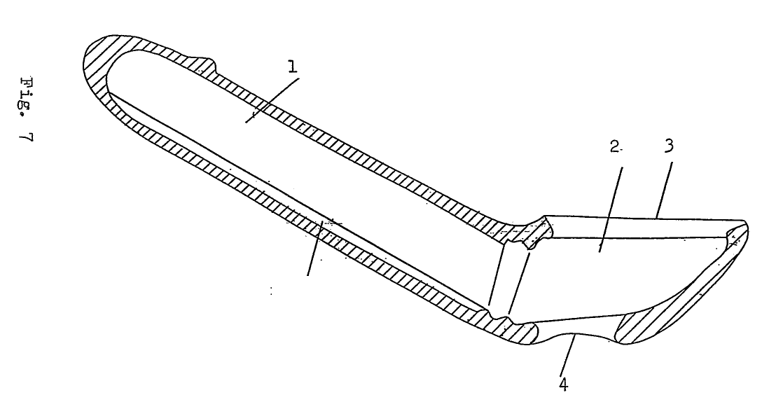

The FIG. 5 shows the front view of the embodiment of the appliance provided with penis ring in longitudinal section and

The FIG. 6 shows a the front view of the version provided with internal ribs in longitudinal section.

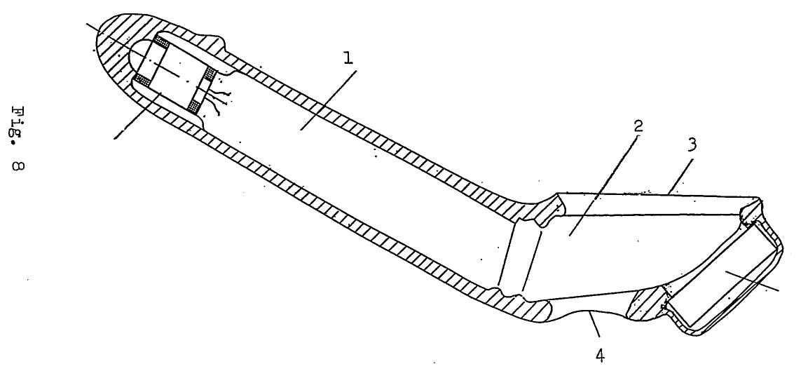

The FIG. 7 shows the front view of the embodiment provided with a longitudinal urethra holder groove in longitudinal section, and

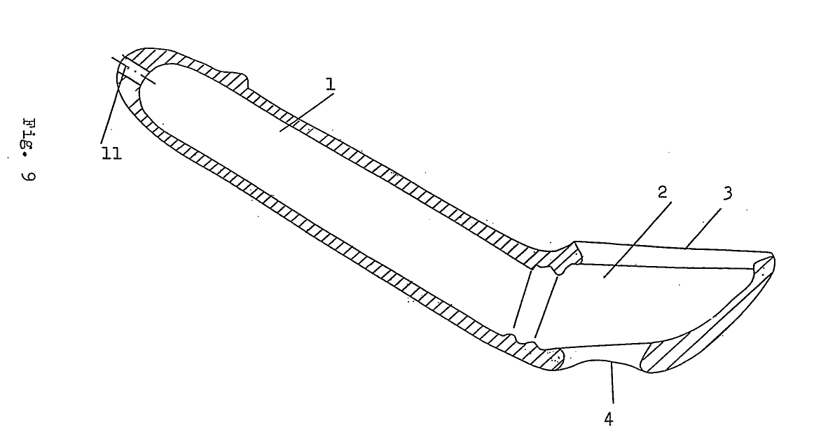

The FIG. 8 shows the front view of such a version, where the appliance is provided with vibrator and battery holder in longitudinal section.

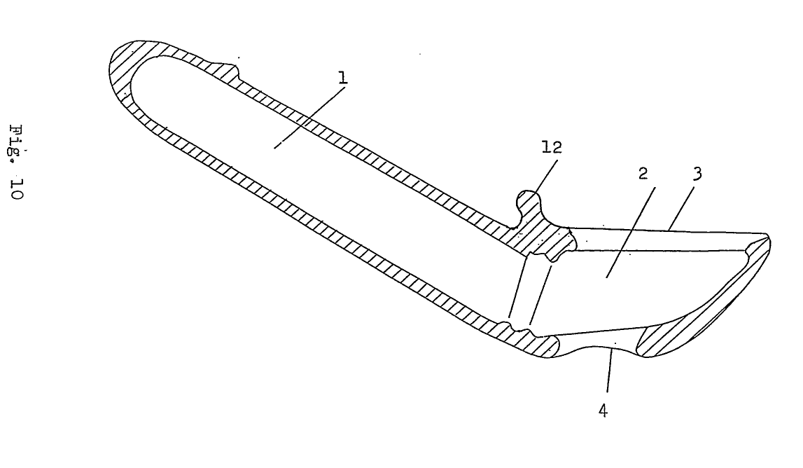

The FIG. 9 shows the longitudinal section of front view of such an embodiment where the appliance is provided with an outlet hole for seminal fluid.

The FIG. 10 shows an embodiment where the outer surface of the appliance is provided with a clitoris exciting bulge.

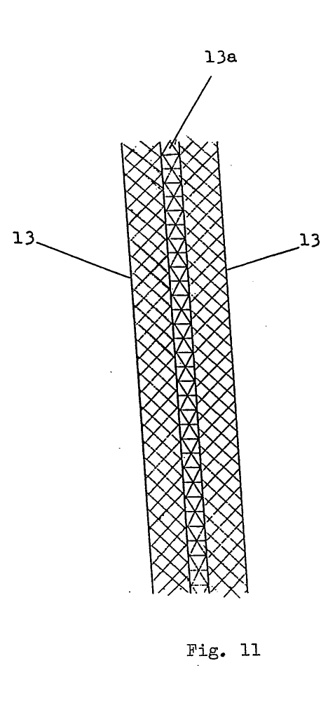

The FIG. 11 shows a section of the wall of a possible embodiment of the appliance.

The penis holder 1 having the shape and dimensions of the penis and ending with a rounded point connected by continuous material transition to the scrotum holder 2, which is provided with an elliptic supporting opening 3 on the upper side and a scrotum passing opening 4 on the lower side are shown in longitudinal section of the front view in the FIG. 1.

The scrotum holder 2 serves to put in and hold the testes, the testes are put in the appliance through the supporting opening 3 and they go out of the appliance through the scrotum passing opening.

The front surface of the flange of the elliptic supporting opening 3 leans against the front wall of the side-bone (the symphysis), the lateral surfaces of the flange surround the scrotum and the rear surface of the flange leans against the rear part of the side-bone before the rectum.

The FIG. 2 shows the embodiment according to the FIG. 1 in top view where the penis holder 1, the elliptic supporting opening 3 on the upper side of the scrotum holder 2 and the scrotum passing opening 4 on the lower side are shown.

The section of the front view according to the FIG. 3 shows a such version of the embodiment according to the FIG. 1 where the outer surface of the penis holder 1 is provided with irregularly distributed superficial protuberances 5.

The FIG. 4 shows the top view of this embodiment.

The section of the front view according to the FIG. 5 shows a version of the embodiment according to the FIG. 1 where the internal surface of the penis holder is provided with a penis ring 6 at the root of the penis.

The penis ring 6 has the function to cause congestion in the penis and keep it in the state of congestion for a longer time.

The section of the front view according to the FIG. 6 shows a version of the embodiment according to the FIG. 1 where the inner surface of the penis holder is provided with regularly arranged circumferential ribs 7.

There are other embodiments wherein the ribs 7 do not extend to the whole circumference but they are arranged only on certain sections of the inner surface of the penis holder 1.

An embodiment with irregularly distributed ribs is also possible.

A version provided with a longitudinal urethra holding groove 8 on the lower side of the internal surface of the penis holder 1. This embodiment serves for the better supporting of the penis.

The FIG. 8 shows a version of the embodiment according to the FIG. 1 wherein the penis holder 1 is provided inside with a vibrator holder 9 in the end with point and the scrotum holder 2 is provided with a battery holder 10.

The above mentioned embodiment is such a version wherein the vibrator having connected with the battery raises the excitement reached by the appliance in known way by vibration.

The FIG. 9 shows a section of front view of such a version of the embodiment according to the FIG. 1 wherein the penis holder 1 is provided with an outlet hole for seminal fluid 11.

Any one of the above mentioned embodiments may be completed with the outlet hole for seminal fluid 11, which serves to let the seminal fluid flow out and improves the feeling of comfort in this way.

The FIG. 10 shows a section of front view of such a version of the embodiment according to the FIG. 1 wherein the penis holder 1 is provided with a clitoris exciting bulge 12 on the outer surface of the penis holder 1 at the root of the penis.

Any one of the above mentioned embodiments may be completed with the clitoris exciting bulge 12, which has the function to improve the excitement of the partner.

The appliance according to the invention is advantageously made of silicon rubber and the superficial protuberances 5, the penis ring 6, the ribs 7 and the clitoris exciting bulge 12 are advantageously also made of silicon rubber.

The FIG. 11 shows the section of an embodiment of the elastic wall of the appliance where the wall is made of a composite material consisting of a plastic net layer 13a (advantageously made of polyamide net) between two silicon rubber layers 13.

A wall structure of such type provides greater strength to the appliance keeping the same elasticity of the wall.

The advantages of the appliance are as follows:

It fits ideally to a penis of any dimensions due to the walls made of soft silicon rubber (A wall thickness of 1 to 5 mm is advantageous).

Its outer shape anatomically imitates the penis.

The self-supported embodiment provides complete freedom of movement.

The shaping of its outer surface anatomically imitates the transmission of excitation.

It solves all the problems of erection, because it enables to perform the sexual act even in case of complete impotence or in the faint state of the penis.

It supports the penis at the side bone both the front and the back sides equally.

It may have several profiles of internal shape, which enables the perfect leading of excitation and improve the blood supply of the corpus cavernosum and makes the erection natural in this way.

It may be applied also as preservative.

It protects against the infections.

The embodiment provided with outlet hole for seminal fluid 11 the pregnancy may come into being in natural way by sexual act.

Its outer and inner shape together imitate the healthy human penis.

We describe the therapeutic-sexual appliance with reference to the example below.

### EXAMPLE 1

The appliance made of elastic silicon rubber has a penis holder 1 with rounded pointed end shaped similarly to the corona penis.

The outer surface of the penis holder 1 has veining and wrinkles imitating the anatomy of the penis.

The penis holder 1 is connected by a continuous material transition to the scrotum holder 2, which has an elliptic supporting opening 3 on its upper side and a scrotum passing opening 4 on its lower side.

An outlet hole for seminal fluid 11 is shaped on the rounded pointed end of the penis holder 1.

There are irregularly distributed superficial protuberances 5 on the outer surface of the penis holder 1 and regularly distributed circumferential ribs 7 on the inner surface of the penis holder 1.

The therapeutic-sexual appliance is manufactured as follows:

A hollow master-pattern is made advantageously of one-component silicon rubber, which contains all the elements of the appliance.

An opening casting form with removable core is made of polyester resin on the base of the master-pattern, and the casting mould is provided with inlet channel, overflow channel and air outlet channel.

Silicon rubber able for cross-linkage is poured into the casting form The form is opened after cross-linking of the silicon rubber and the therapeutic-sexual appliance is taken out together with the core.

The therapeutic-sexual appliance is stripped off from the core

### EXAMPLE 2

The embodiment according to Example 1 with the difference that a longitudinal urethra holding groove 8 is also shaped on the inner surface of the penis holder 1 and it is provided also with a clitoris exciting bulge 12 on the outer surface.

The walls of the therapeutic-sexual appliance is made of a composite material in such a way that silicon rubber 13 able to cross-linking is applied onto the internal core described in the Example 1 then a plastic net layer 13a is put onto the surface, which is still soft.

The semi-finished product with the core is put then into the casting form described in the Example 1, silicon rubber layer is applied onto it then the material of the product is cross-linked in the casting form.

The rest part of process is done as in the Example 1.

The method how to put on the therapeutic-sexual appliance is described below.

The appliance is turned inside out and put on the penis in any state of erection in such a way that first the prepuce is drawn back and the corona penis is put to the tipped end of the appliance then the appliance is continuously rolled towards the root of the penis.

The part of the penis behind the corona should be advantageously constricted and the appliance is rolled towards the scrotum.

When the scrotum is reached the user pulls the testes through the opening for the testes shaped on the rear part of the appliance. The penis is now in the therapeutic-sexual appliance and in a normally erected status anatomically capable for sexual act.

It is advantageous to lubricate the outer surface of the therapeutic-sexual appliance with a lubricating gel (e.g. with a créme, Vaseline or a lubricating gel).

The advantages provided by the therapeutic-sexual appliance are as follows:
Men can use it in such a way that it does not require any complicated fastening at all.
Having self-supporting structure it may be used in any state of erection of the penis (even in languid state) in contrary to the condom used in known way.
It may be applied to, it fits closely to a penis of any dimensions.
It is simple to use.
The elastic soft wall and the shape of outer surface contributes to the feeling of body, the texture of the surface anatomically imitates the transmission of excitement.
It does not hinder men in the sexual freedom by the feeling of bad comfort, protects against infections and protects against undesired pregnancy in the case of necessity, as well.

### LIST OF REFERENCES

1 penis holder
2 scrotum holder
3 supporting opening
4 scrotum passing opening
5 superficial protuberance
6 penis ring
7 circumferential rib
8 urethra holding groove
9 vibrator holder
10 battery holder
11 outlet hole for seminal fluid
12 clitoris exciting bulge
13 silicon rubber layer
13a plastic net layer

1. A self-supporting hollow therapeutic-sexual appliance for men characterized by that it has a penis holder having shape and surface similar to the penis and ending with a rounded point, a scrotum holder connected to it by continuous material transition provided with an elliptic supporting opening on the upper side and with a scrotum passing opening on the lower side.

2. The therapeutic-sexual appliance according to the claim 1 characterized by that the outer surface of the penis holder is provided with either regularly or irregularly distributed superficial protuberances.

3. The therapeutic-sexual appliance according to claim 1 characterized by that the penis holder is provided with a penis ring at the root of the penis.

4. The therapeutic-sexual appliance according to claim 1 characterized by that the inner surface of the penis holder is provided with either regularly or irregularly distributed ribs.

5. The therapeutic-sexual appliance according to claim 1 characterized by that the penis holder is provided with a longitudinal urethra holding groove in the inner surface at the bottom.

6. The therapeutic-sexual appliance according to claim 1 characterized by that the penis holder is provided with a vibrator holder in the part ending with rounded point and the scrotum holder is provided with a battery holder inside.

7. The therapeutic-sexual appliance according to claim 1 characterized by that the penis holder is provided with an outlet hole for the seminal fluid on the rounded pointed end.

8. The therapeutic-sexual appliance according to claim 1 characterized by that the penis holder is provided with a clitoris exciting bulge on the outer surface at the root of the penis.

9. The therapeutic-sexual appliance according to claim 1 characterized by that its elastic wall is made of silicon rubber.

10. The therapeutic-sexual appliance according to claim 1 characterized by that its elastic wall is made of a composite material wherein a plastic net layer is placed between two silicon rubber layers.
